(19) United States
(12) Patent Application Publication
(10) Pub. No.: US 2024/0108614 A1
(43) Pub. Date: Apr. 4, 2024

(54) Methods of Treating Diabetic Cardiomyopathy by Inhibiting GLUT1

(71) Applicant: University of Cincinnati, Cincinnati, OH (US)

(72) Inventors: Konstantinos Drosatos, Cincinnati, OH (US); Md Sobuj Mia, Cincinnati, OH (US)

(73) Assignee: University of Cincinnati, Cincinnati, OH (US)

(21) Appl. No.: 18/376,187

(22) Filed: Oct. 3, 2023

Related U.S. Application Data
(60) Provisional application No. 63/412,719, filed on Oct. 3, 2022.

(51) Int. Cl. A61K 31/4709; A61K 31/12; A61K 31/155; A61K 31/235; A61K 31/351; A61K 31/426; A61K 31/4406; A61K 31/64; A61K 38/26; A61K 38/28; A61K 45/06; A61P 3/10; A61P 9/04

(52) U.S. Cl. CPC A61K 31/4709; A61K 31/12; A61K 31/155; A61K 31/235; A61K 31/351; A61K 31/426; A61K 31/4406; A61K 31/64; A61K 38/26; A61K 38/28; A61K 45/06; A61P 3/10; A61P 9/04

(57) ABSTRACT

Provided herein are methods for treating diabetic cardiomyopathy in a patient in need thereof, such as a patient diagnosed with type 1 or type 2 diabetes. The methods generally include administering a therapeutically effective amount of one or more glucose transporter isoform 1 (GLUT1) inhibitors to the patient. Exemplary GLUT1 inhibitors include STF-31, WZB117, phloretin, and BAY-876.

### CROSS-REFERENCE TO RELATED APPLICATIONS

This application claims the benefit of U.S. Provisional Application No. 63/412,719 filed Oct. 3, 2022, the entire disclosure of which is incorporated herein by reference in its entirety.

### TECHNICAL FIELD

The present disclosure relates generally to the field of medical therapeutics, specifically compositions and methods for treating diabetic cardiomyopathy.

### BACKGROUND

Diabetes will affect 10% of the global population by 2040. Diabetic cardiomyopathy is a major complication in diabetes, accompanied by altered cardiac energetics, impaired mitochondrial function, and oxidative stress. Diabetic cardiomyopathy occurs without coronary artery disease, hypertension, and valvular disease. No specific treatment is currently available for diabetic cardiomyopathy. Current treatment protocol involves intense glycemic control, possibly in combination with other therapeutic agents, such as ACE inhibitors or beta blockers.

The primary cause of diabetic cardiomyopathy remains elusive. Pre-clinical findings that attributed causality to reactive oxygen species and oxidative stress were not supported by clinical trials. Other studies have attempted to correlate diabetic cardiomyopathy with cardiac accumulation of lipids, such as ceramides and diacylglycerols. While both human and animal heart failure studies report that glucose is a preferable fuel at the expense of fatty acid oxidation during ischemia and hypertrophy, the opposite occurs in diabetic hearts. In contrast to the failing heart, fatty acids are the preferred substrate in a healthy heart, though glucose metabolism is also enhanced to support oxidative phosphorylation. In diabetic hearts, the heart maintains its preference for lipid oxidation as an electron source for ATP synthesis.

Still other studies have attempted to correlate the use of glucose-lowering therapies, such as SGLT2 inhibition using dapagliflozin or empagliflozin, with their reported cardioprotective properties, primarily believed to result from offloading glucose.

Cardiac glucose uptake is mediated by insulin-dependent and insulin-independent mechanisms. Glucose transporter isoform 1 (GLUT1) is insulin-independent and is a membrane protein that mediates facilitated diffusion of glucose into the cell. Glucose transporter isoform 4 (GLUT4) is insulin-dependent and is responsible for the majority of glucose transport. Specifically, insulin triggers GLUT4 translocation to the cell membrane for mediating most cardiac glucose uptake. Both type 1 diabetes and type 2 diabetes impair GLUT4 translocation. For this reason, diabetic cardiomyopathy has been associated with abnormalities mostly of cardiac lipid metabolism and less of glucose metabolism.

Kruppel-like factor 5 (KLF5), also known as intestinal enriched Kruppel-like factor (IKLF) or basic transcription element binding protein 2 (Bteb2), has been shown to regulate cardiac fatty acid oxidation via direct activation of PPARα (peroxisome proliferator—activated receptor α). Previous studies indicate that diabetes is associated with increased cardiac expression of KLF5 and PPARα that regulate cardiac lipid metabolism, as well as the regulation of KLF5 expression by the transcriptional factor FOXO1 (Kyriazis, et. al. Circulation Research. 2021; 128:335-357). However, prior to the instant disclosure, the relationship between GLUT1, KLF5, and diabetic cardiomyopathy has not been appreciated.

Accordingly, a need exists to identify the pathophysiological mechanisms by which diabetic cardiomyopathy occurs and to develop specific treatments that can treat and/or prevent diabetic cardiomyopathy, and reduce mortality and morbidity associated therewith.

### SUMMARY

The instant disclosure elucidates the pathological mechanism of cardiac uptake of glucose via GLUT1 and the effects of glucose accumulation on FOXO1 and its target genes in cardiotoxicity and diabetic cardiomyopathy. Specifically, the instant disclosure demonstrates that glucose uptake via GLUT1 is a central pathological event that activates various pathways, including lipotoxicity, and accounts for diabetic cardiomyopathy. Accordingly, provided herein are methods for the treatment of diabetic cardiomyopathy comprising the administration of GLUT1 inhibitors.

Example embodiments disclosed herein are directed to methods of treating diabetic cardiomyopathy in a patient in need thereof, the method comprising administering to the patient a therapeutically effective amount of one or more glucose transporter 1 (GLUT1) inhibitors, such as STF-31, WZB117, phloretin, and BAY-876. In some embodiments, the GLUT1 inhibitor is STF-31.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIGS. 1A-1F demonstrate that cardiac glucose utilization is increased in early type 1 diabetes. FIG. 1A graphically illustrates a box and whisker plot for myocardial glucose values in late type 1 diabetes compared with non-diabetic controls. FIG. 1B graphically illustrates the mitofuel seahorse analysis for substrate dependence of adult cardiomyocytes in early type 1 diabetes. FIG. 1C graphically illustrates the mitofuel seahorse analysis for substrate dependence of adult cardiomyocytes in early type 1 diabetes. FIG. 1D graphically depicts the glucose tolerance. FIG. 1E depicts the cardiomyocyte dependence on fatty acids. FIG. 1F depicts the cardiomyocyte dependence on glucose.

FIGS. 2A-2E demonstrate that FOXO1 deacetylation occurs in late T1D and high glucose levels increased SIRT1 and SIRT1-FOXO1 protein-protein interaction. Cardiac expression of various FOXO1 targets, such as SIRT1, pyruvate dehydrogenase kinase-4 (PDK4), Bc1-2-like protein 11 (Bim), catalase (Cat) and heme oxygenase-1 (Ho1) were not increased in early type 1 diabetes (FIG. 2A) but were higher in late type 1 diabetes (FIG. 2B). High glucose stimulated the expression of FOXO1 targets, SIRT1, KLF5, and BIM (FIG. 2C). Treatment of AC16 cells with high glucose alone lowered insulin-mediated phosphorylation of AKT (FIG. 2E) and FOXO1 (FIG. 2D), demonstrating nuclear retention of FOXO1.

FIGS. 3A-3B demonstrate that pharmacological inhibition of GLUT1 prevents cardiac dysfunction in early type 1 diabetes. FIG. 3A-3B depict the plasma glucose levels (FIG. 3B), and the mitral valve E/A ratio (FIG. 3A) in healthy and type 1 diabetic mice, treated with either STF-31 or control vehicle. Diabetic mice treated with ST-F31 did not develop diastolic dysfunction (FIG. 3A) despite persistent hyperglycemia (FIG. 3B.)

FIGS. 4A-4E demonstrate that long-term GLUT1 inhibition prevents and restores cardiac dysfunction and KLF5 upregulation. Diabetic mice treated with ST-F31 did not develop either systolic (FIG. 4B) or diastolic dysfunction (FIG. 4C) despite persistent hyperglycemia (FIG. 4A). In another mouse cohort, treatment with either ST-F31 or dapa started after cardiac dysfunction was confirmed (6 weeks of type 1 diabetes) and continued until the twelfth week. This treatment corrected both systolic (FIG. 4D) and diastolic (FIG. 4E) dysfunction.

FIGS. 5A-5D demonstrate that pharmacologic inhibition of GLUT1 is cardioprotective in a mouse model of Type-2 diabetes. Diabetic mice treated with STF-31 did not develop either systolic (FIG. 5B) or diastolic dysfunction (FIG. 5C) despite persistent hyperglycemia (FIG. 5A). Further, KLF5 upregulation was also prevented (FIG. 5D).

FIGS. 6A-6E demonstrate that cardiomyocyte-specific GLUT1 genetic deletion reduces glucose uptake and prevents diabetic cardiomyopathy in type 1 diabetes. These results demonstrate a significant reduction in glucose uptake compared with the diabetic mice with GLUT1 (FIG. 6A). FIG. 6B depicts a PET scan, demonstrating the reduction in glucose uptake. The diabetic knockout mice did not develop either systolic (FIG. 6C) or diastolic dysfunction (FIG. 6D). Further, KLF5 upregulation was also prevented (FIG. 6E).

FIGS. 7A-7D demonstrate that cardiomyocyte-specific GLUT1 genetic deletion is cardioprotective in a mouse model of Type-2 diabetes, showing that the diabetic knockout mice did not develop either systolic (FIG. 7B) or diastolic dysfunction (FIG. 7C) despite persistent hyperglycemia (FIG. 7A). Further, KLF5 upregulation was also prevented (FIG. 7D).

### DETAILED DESCRIPTION

The details of one or more embodiments of the presently disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

The terms “administer,” “administration,” and “administering,” as used herein, refers to any route of administering an effective amount of a therapeutic agent. In embodiments, the administering includes, but is not limited to, intravenous, intraperitoneal, intrapericardial, intramyocardial, intracoronary, subcutaneous, intramuscular, oral, intracerebral, intraspinal, intrathecal, subarachnoid, epidural, periocular, intraocular administration, and the like. In some embodiments, the therapeutic agent is administered intramuscularly, subcutaneously, or intravenously.

The terms “treat,” “treatment,” and “treating,” as used herein, refer to a method of alleviating or abrogating a disease, disorder, and/or symptoms thereof in a subject, including a mammal. In certain embodiments, the subject is a human patient.

Glucose transporters facilitate the transport of glucose across the plasma membrane. The present inventors investigated the effects of glucose transporter 1 (GLUT1) inhibition on diabetic cardiomyopathy, particularly the role of GLUT1 in cardiac function.

The present disclosure demonstrates that, mechanistically, glucose activates a SIRT1-FOXO1-KLF5 signaling axis, aggravating glucotoxicity and lipotoxicity, thereby leading to diabetic cardiomyopathy. The instant disclosure also elucidates the role of GLUT1 in cardiac uptake of glucose, identifying it as a molecular target for the treatment of diabetic cardiomyopathy.

Moreover, GLUT1-specific pharmacological inhibition is shown herein to prevent and ameliorate cardiac dysfunction and reduce oxidative stress, in diabetic cardiomyopathy, even with persistent hyperglycemia. This is surprising for several reasons. First, prior to the instant disclosure, GLUT4 was believed to be the primary glucose transporter isoform in cardiac uptake of glucose. However, the present inventors have demonstrated that GLUT1 is at least as important as GLUT4 in this role. Further, upregulation and/or overexpression of GLUT1 have previously been shown to have cardioprotective effects. Conventional treatment of diabetic cardiomyopathy relies on glucose

Specifically, the instant disclosure demonstrates that glucose uptake via GLUT1 is a central pathological event that activates various pathways, including lipotoxicity, and accounts for diabetic cardiomyopathy. Further, the instant disclosure elucidates the role of GLUT1 inhibition in cardioprotection, more specifically, in preventing glucose uptake into the heart. This disclosure also elucidates key differences between early and late type 1 diabetes, including unexpected differences in myocardial glucose values and transcriptional activation of FOX01. Accordingly, provided herein are methods for the treatment of diabetic cardiomyopathy comprising the administration of GLUT1 inhibitors.

In some embodiments, the methods described herein comprise administering one or GLUT1 inhibitors to a patient suffering from diabetic cardiomyopathy. As used herein, “GLUT1 inhibitor,” refers to an agent that reduces the expression level and/or activity of GLUT1. The GLUT1 inhibitor can inhibit GLUT1 via any suitable mechanism, including direct inhibition, binding inhibition, allosteric modulation, coactivator and/or corepressor interference, downstream pathway inhibition, and/or siRNA and/or antisense nucleotide degradation.

Non-limiting examples of GLUT1 inhibitors include STF-31, WZB117, phloretin, BAY-876, fasentin, apigenin, genistein, phloretin, naringenin, resveratrol, oxime-based GLUT1 inhibitors, pyrrolidine-derived GLUT1 inhibitors, DRB18 and combinations thereof. In some embodiments, the GLUT1 inhibitor is selected from the group consisting of STF-31, WZB117, phloretin, and BAY-876. In a very specific embodiment, the GLUT1 inhibitor is STF-31.

Administering the GLUT1 inhibitor includes systemic and non-systemic administration. In some embodiments, systemic administration includes enteral administration and/or parenteral administration. In some embodiments, systemic administration includes targeted drug delivery of the GLUT1 inhibitor to the heart, such as with lipid nanoparticles, exosomes, and the like.

In some embodiments, non-systemic administration includes intracoronary delivery, intrapericardial delivery, and/or intramyocardial delivery via transepi cardi al and/or transendocardial routes. In some embodiments, non-systemic administration involves a pump, wafer, cardiac patch and/or microbubble cavitation.

In some embodiments, a therapeutically effective amount of the GLUT1 inhibitor is a dose from about 0.1 mg/kg body weight to about 100 mg/kg body weight, including about 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, and 99 mg/kg, or any range having endpoints defined by any two of the aforementioned values.

In some embodiments, administering the GLUT1 inhibitor comprises administering a plurality of doses to the patient, such as two doses, three doses, four doses, etc. In some embodiments, the plurality of doses are spaced at specific time points, such as hourly, twelve times a day, eight times a day, six times a day, four times a day, three times a day, twice a day, daily, every other day, three times a week, twice weekly, weekly, biweekly, monthly, etc. In some embodiments, the GLUT1 inhibitor is administered beginning after a patient is diagnosed with diabetes, such as substantially at the time of diagnosis, a day after diagnosis, a week after diagnosis, a month after diagnosis, or any time period after diagnosis. In some embodiments, the GLUT1 inhibitor is administered to the patient at the onset of one or more additional therapies, such as diabetic medication. In other embodiments, the GLUT1 inhibitor is administered after a patient has confirmed cardiac dysfunction, such as substantially at the time of confirmation, a day after confirmation, a week after confirmation, a month after confirmation, etc. That is, in certain embodiments, the GLUT1 inhibitor is administered to a patient after diagnosis of cardiac dysfunction. In some embodiments, administration of the GLUT1 inhibitor is discontinued after the cardiac dysfunction resolves. In a more specific embodiment, the patient is a diabetic patient suffering from type 1 or type 2 diabetes.

Data reported herein indicate that, despite similar blood glucose levels in early type 1 diabetes and late type 1 diabetes, myocardial glucose levels remained low in early type 1 diabetes. Only in late type 1 diabetes did the myocardial glucose values rise. Furthermore, this disclosure demonstrates that KLF5 activation, which relies on GLUT1-mediated glucose uptake, increases further GLUT1 expression, thus contributing to a vicious cycle of cardiac glucose toxicity.

Pharmacological, as well as CM-specific GLUT1 inhibition preserved and restored cardiac function, despite hyperglycemia. In early type 1 diabetes, pharmacological inhibition of GLUT1 prevented cardiac dysfunction. Long-term GLUT1 inhibition prevented cardiac dysfunction and KLF5 upregulation. Treatment of diabetic mice with STF-31 after the onset of cardiomyopathy corrected cardiac function. Genetic ablation of cardiomyocyte GLUT1 diminished glucose uptake and prevented diabetic cardiomyopathy.

In embodiments, the methods disclosed herein further comprise administering to the subject an effective amount of a secondary therapeutic agent. The selection of a second therapeutic agent will depend on the clinical needs of the subject. In embodiments, the primary therapeutic agent and the secondary therapeutic agent are administered consecutively or concurrently. The primary therapeutic agent(s) and the secondary therapeutic agent(s) may be administered in separate dosage forms or in the same unit dosage.

Non-limiting examples of secondary therapeutic agents include antiplatelet medications (such as aspirin, clopidogrel, ticagrelor, prasugrel, dipyridamole, ticlopidine, and eptifibatide), anticoagulant medications (such as tissue plasminogen activator (tPA), heparin, warfarin, rivaroxaban, dabigatran, apixaban, edoxaban, enoxaparin, and fondaparinux), anti-inflammatory agents, vasodilators (such as nitrates, ACE inhibitors, angiotensin receptor blockers, calcium channel blockers, sildenafil, magnesium-containing vasodilators, and the like), beta blockers, cholesterol-lowering agents, mineralocorticoid receptor antagonists (such as spironolactone, eplerenone, finerenone, the like) ranolazine, SGLT2 inhibitors (such as empagliflozin and dapagliflozin), GLP1 receptor agonists (such as semaglutide, dulaglutide, lixisenatide, and liraglutide), incretin mimetics, insulin, alpha-glucosidase inhibitors, biguanides (such as metformin), bile acid sequestrants, dopamine-2 agonists, gliptins, glinides, sulfonylurea, thiazolidinedione, and combinations thereof.

### EXAMPLES

The following Examples are offered by way of illustration and are presented in a manner such that one skilled in the art should recognize are not meant to be limiting to the present disclosure as a whole or to the appended claims.

### Example 1: Cardiac Glucose Utilization is Increased in Early Type 1 Diabetes

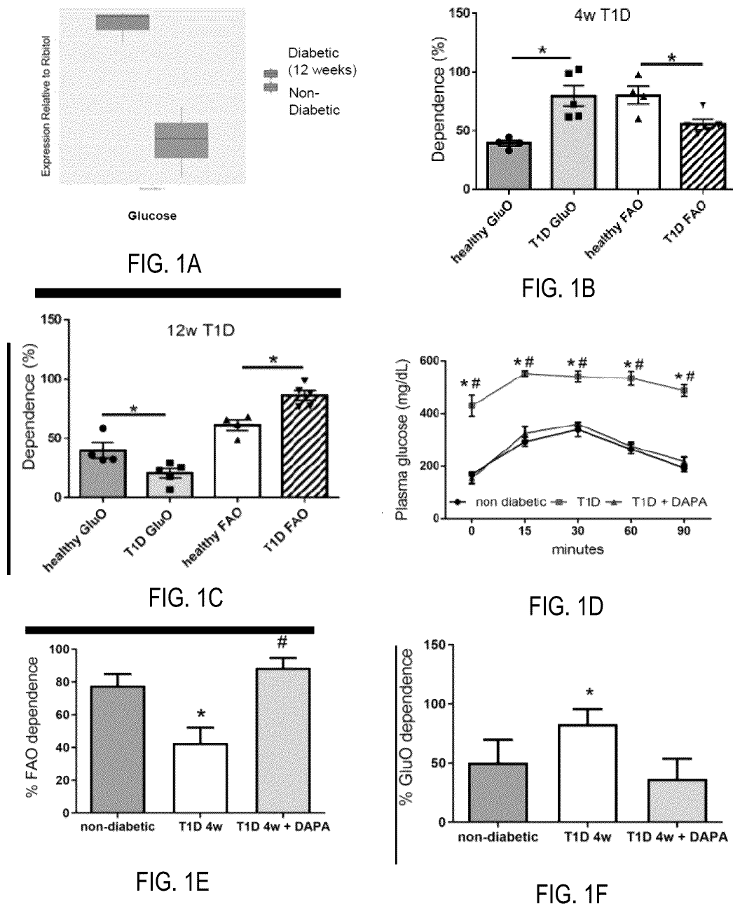

The present inventors demonstrated that myocardial glucose is elevated in late type 1 diabetes (12 weeks after induction of diabetes) compared with healthy mice (FIG. 1A) and early type 1 diabetes (4 weeks after induction of diabetes), despite relatively similar levels of hyperglycemia.

To determine this, the cardiac lipidome in healthy mice, mice with early type 1 diabetes, mice with late type 1 diabetes, and mice with early type 1 diabetes that had been treated with an SGLT2 inhibitor (dapa) was evaluated (data not shown). No significant changes were observed between the healthy mice, the mice with early type 1 diabetes, and the mice treated with dapa. Because of this, the involvement of glucose metabolism pathways in the onset and progression of diabetic cardiomyopathy was investigated.

Metabolomic analyses showed higher cardiac glucose content (FIG. 1A) and lower glucose-derived one-carbon metabolites, such as serine and glycine, in late type 1 diabetes. Myoinositol, which is inversely correlated with hyperglycemia and cardiac triglyceride accumulation, was reduced in late type 1 diabetes. Krebs cycle intermediates in late type 1 diabetes were also reduced, demonstrating increased oxidation compared to non-diabetic mice. Aspartate, a metabolite in the malate-aspartate shuttle, which moves NADH into mitochondria to generate ATP, is also decreased in late type 1 diabetes.

Urea and N-acetylglutamic acid availability was decreased in early type 1 diabetes, demonstrating that acetyl-CoA and glutamate are not available to produce N-acetylglutamic acid for the reduction of ammonia to urea. Mice with early type 1 diabetes that were treated with dapa showed lower cardiac levels of glucose-derived metabolites, such as lactate, methionine, tyrosine, and phenylalanine, demonstrating that dapa lowers cardiac glucose content in early type 1 diabetes. Thus, improvement of cardiac function by dapa in early type 1 diabetes is associated with lower glucose metabolites, while diabetic cardiomyopathy in late type 1 diabetes is accompanied by higher myocardial glucose content.

To explain the observed lower myocardial glucose content in early type 1 diabetes compared to late type 1 diabetes, despite similar hyperglycemia in both stages, mitochondrial glucose utilization was assessed in both stages. Seahorse analysis for fatty acid and glucose dependence in isolated primary cardiomyocytes from mice with early and late type 1 diabetes showed that in early type 1 diabetes, cardiomyocytes rely on glucose rather than fatty acids compared to cardiomyocytes isolated from healthy mice (FIG. 1B). Conversely, fatty acid dependence is higher in cardiomyocytes of mice obtained in late type 1 diabetes. (FIG. 1C). This difference was accompanied by a trend for lower expression of pyruvate dehydrogenase kinase-4 (PDK4) in early type 1 diabetes and significantly higher expression in late type 1 diabetes (data not shown), which suggests inhibition of pyruvate conversion to acetyl-CoA in late type 1 diabetes. Thus, mice with early type 1 diabetes show higher mitochondrial utilization of glucose in cardiomyocytes, which declines during progression to late type 1 diabetes at the expense of higher fatty acid usage. This is reflected in the low myocardial glucose content in early type 1 diabetes and high myocardial glucose content in late type 1 diabetes.

Treatment of diabetic mice with dapa, normalized plasma glucose levels and glucose tolerance (FIG. 1D) without correcting plasma insulin levels. It also restored fatty acid as the primary fuel for cardiomyocytes (FIG. 1E), while it decreased glucose dependence (FIG. 1F). The cardiomyocyte dependence shift from glucose to fatty acid in early type 1 diabetes was accompanied by improvement of systolic and diastolic cardiac function. Lower plasma lipid levels in diabetic mice treated with dapa, in combination with unchanged secretion of very low-density-lipoprotein-associated triglycerides (data not shown) suggests normalization of systemic and cardiac lipid mobilization and consumption. Both adipose tissue lipolysis and cardiac acyl-carnitine levels were restored to normal levels in diabetic mice treated with dapa. Thus, cardiac dysfunction of mice in early type 1 diabetes is associated with lower fatty acid utilization and increased glucose dependence.

### Example 2: FOXO1 Deacetylation Occurs in Late T1D

The identification that higher glucose utilization was associated with the onset of cardiac dysfunction led to the investigation of signaling pathways that are triggered by the early to mid-stage metabolic changes and cause cardiomyopathy in late type 1 diabetes. The present inventors have previously shown that neither cardiac KLF5 expression nor PPARα expression are increased in early type 1 diabetes and that FOXO1 and KLF5 activation occur in late type 1 diabetes and cause diabetic cardiomyopathy.

Activation of the KLF5 promoter is controlled by FOXO1. DNA binding affinity of FOXO1 is increased with deacetylation, which prevents phosphorylation and nuclear export. Insulin signaling inhibition or high glucose content activate FOXO1, which leads to diabetic cardiomyopathy. Surprisingly, the present inventors discovered that, even in early type 1 diabetes, FOXO1 is dephosphorylated, and thus present in the nucleus.

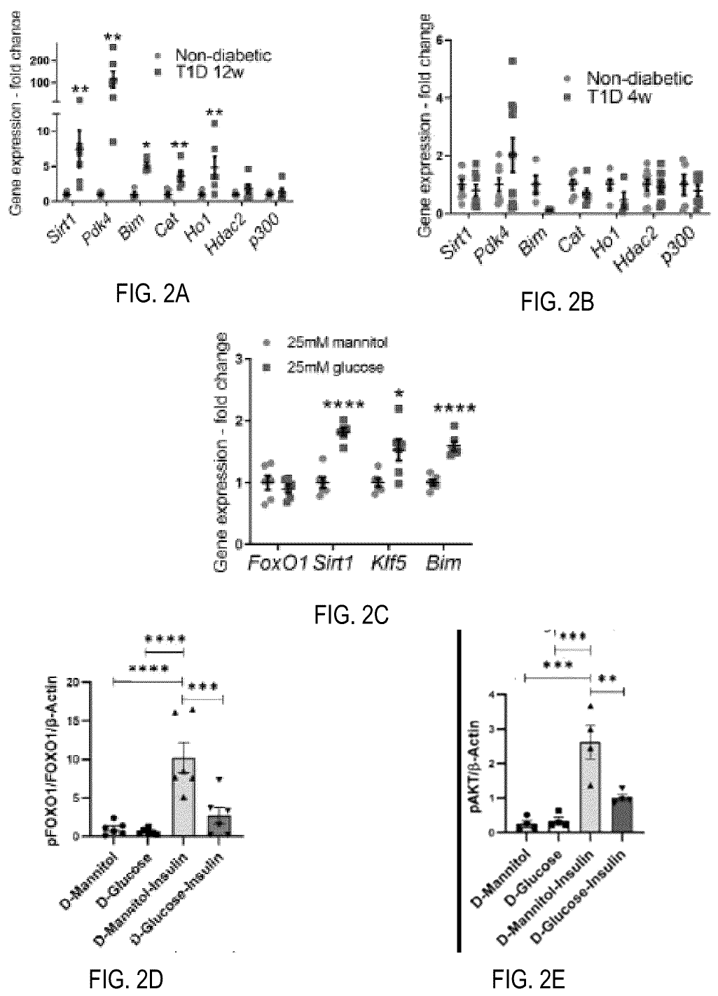

Despite its nuclear localization, FOXO1 is transcriptionally inactive in early type 1 diabetes. Indeed, cardiac expression of various FOXO1 targets, such as SIRT1, pyruvate dehydrogenase kinase-4 (PDK4), Bc1-2-like protein 11 (Bim), catalase (Cat), and heme oxygenase-1 (Ho1) were not increased in early type 1 diabetes, but were higher in late type 1 diabetes. (FIG. 2A, 2B). Thus, FOXO1 activation and KLF5 upregulation occur in late type 1 diabetes when cardiomyocytes do not oxidize as much glucose as in early type 1 diabetes.

The present inventors also investigated whether FOXO1 activation and eventual KLF5 upregulation in late type 1 diabetes are driven by high myocardial glucose content. To explore this human cardiomyocyte cells (AC16) were treated with 25 mM glucose. This high glucose stimulated the expression of FOXO1 targets, SIRT1, KLF5, and BIM (FIG. 2C). Notably, treatment of AC16 cells with high glucose alone lowered insulin-mediated phosphorylation of FOXO1 (FIG. 2D) and AKT (FIG. 2E) demonstrating increased nuclear FOXO1 retention.

The transcriptional activity was confirmed by evaluating cardiac FOXO1 acetylation and SIRT protein levels in mice with early and late type 1 diabetes. These analyses showed no differences between healthy mice and those with early type 1 diabetes, but lower FOXO1 acetylation (meaning more active FOXO1) and higher SIRT1 in late type 1 diabetes. Accordingly cardiac SIRT1 protein levels were unchanged in early type 1 diabetes and higher in late type 1 diabetes (data not shown). Thus, the transition from early to late type 1 diabetes is accompanied by myocardial glucose accumulation which accounts for FOXO1 activation and KLF5 upregulation that eventually leads to cardiac lipotoxicity and diabetic cardiomyopathy.

### Example 3: Pharmacologic GLUT1 Inhibition Prevents Cardiac Dysfunction in Early Type-1 Diabetes Despite Hyperglycemia

As myocardial glucose utilization was critical for the onset of cardiomyopathy in early type 1 diabetes, and glucose accumulation was associated with KLF5 activation and cardiac lipotoxicity in late type 1 diabetes, potential involvement of GLUT in early and late type 1 diabetes was investigated.

To explore whether GLUT1 inhibition holds therapeutic potential against diabetic cardiomyopathy, diabetic mice were treated with a GLUT1 inhibitor (STF-31). Specifically, type 1 diabetes was induced in WT mice and mice were monitored for twelve weeks.

To evaluate the impact of GLUT1 and myocardial glucose content changes on cardiac function and structure, 2D echocardiography was performed to measure EF, FS, end-diastolic and end-systolic volumes (EDV, ESV), stroke volume (SV), and cardiac output (CO) using M-mode short axis images, automatic tracing, myocardial strain and strain rate with speckle tracking. From the pulsed wave Doppler spectral waveforms, the peak early and late diastolic transmitral velocities (E, A waves) were measured to obtain the E/A ratio.

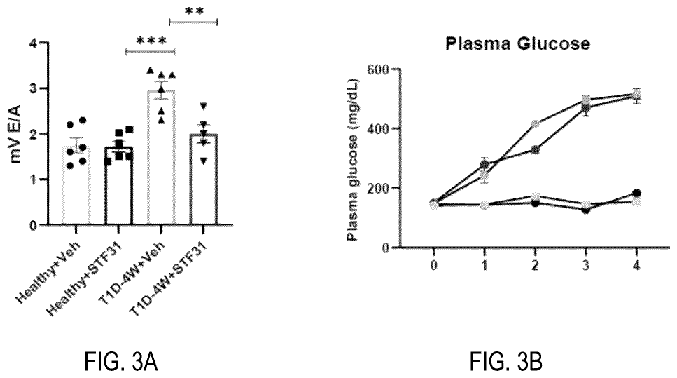

One group of mice was treated with STF-31 for the entire twelve-weeks period. Diabetic mice treated with STF-31 did not develop either systolic or diastolic (FIG. 3A) dysfunction despite persistent hyperglycemia (FIG. 3B).

### Example 4: Long-term GLUT1 inhibition prevents cardiac dysfunction and KLF5 upregulation

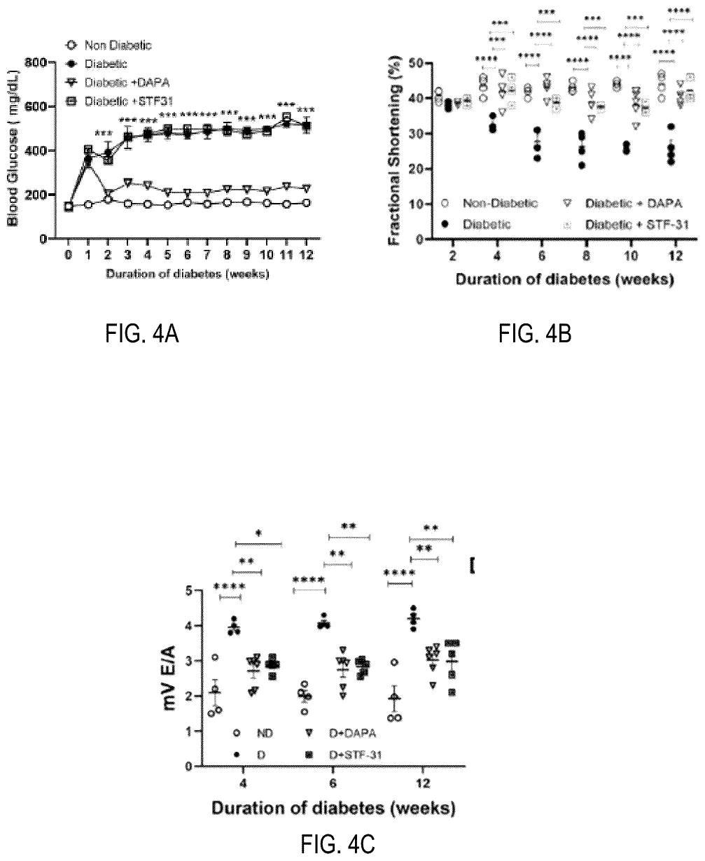

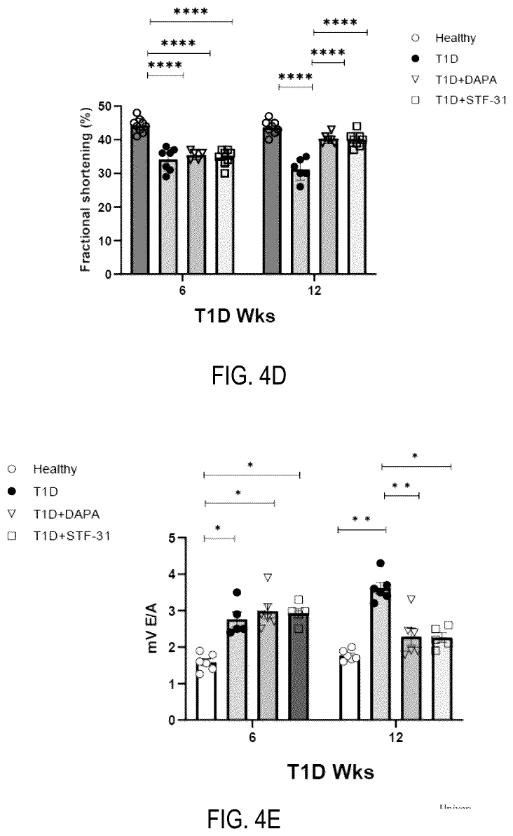

Prior to this disclosure, treatment of diabetic patients with SGLT2 inhibitors (dapa or empagliflozin), has shown the most robust cardioprotective effects. Treatment of diabetic mice with dapa corrected hyperglycemia (FIG. 4A) and prevented systolic (FIG. 4B) and diastolic heart failure (FIG. 4C) in both early and late type 1 diabetes. Interestingly, diabetic cardiomyopathy in early type 1 diabetes was not associated with altered cardiac total lipidome, as noted above, or lipid species that have been linked to cardiac dysfunction.

GLUT1 inhibitors have potential as therapeutics for diabetic cardiomyopathy, as demonstrated by STF-31 in diabetic and non-diabetic mice over the course of twelve weeks. Diabetic mice treated with STF-31 did not develop either systolic (FIG. 4B) or diastolic dysfunction (FIG. 4C) despite persistent hyperglycemia (FIG. 4A).

In another mouse cohort, treatment with either STF-31 or dapa started after cardiac dysfunction was confirmed (6 weeks of type 1 diabetes) and continued until the twelfth week. This treatment corrected both systolic (FIG. 4D) and diastolic (FIG. 4E) dysfunction. Importantly, treatment with either STF-31 or dapa prevented upregulation of KLF5. Thus, inhibition of GLUT1 appears as a novel therapeutic target for diabetic cardiomyopathy that prevents activation of the cardiotoxic FOXO1-KLF5 axis.

### Example 5: Pharmacologic Inhibition of GLUT1 is Cardioprotective in a Mouse Model of Type-2 Diabetes (HFD for 40 Weeks)

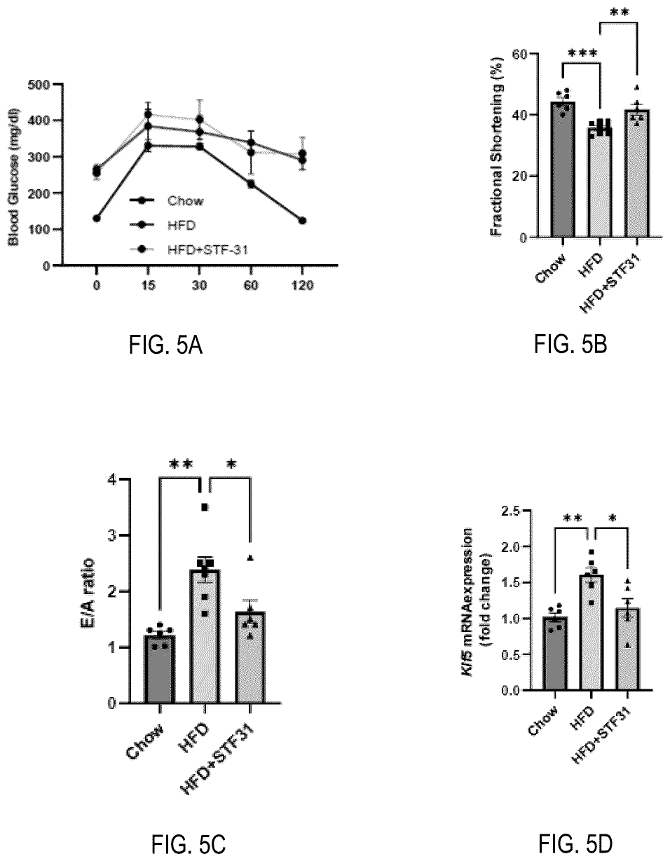

Similar results were shown in mouse models of type 2 diabetes, wherein mice were fed a high-fat diet (HFD) for 32 weeks prior to initiating 8 weeks of treatment with SFT-31. Diabetic mice treated with STF-31 did not develop either systolic (FIG. 5B) or diastolic dysfunction (FIG. 5C) despite persistent hyperglycemia (FIG. 5A). Further, KLF5 upregulation was also prevented (FIG. 5D).

### Example 6: Genetic Ablation of CM-GLUT1 Diminished Glucose Uptake and Prevented Diabetic Cardiomyopathy

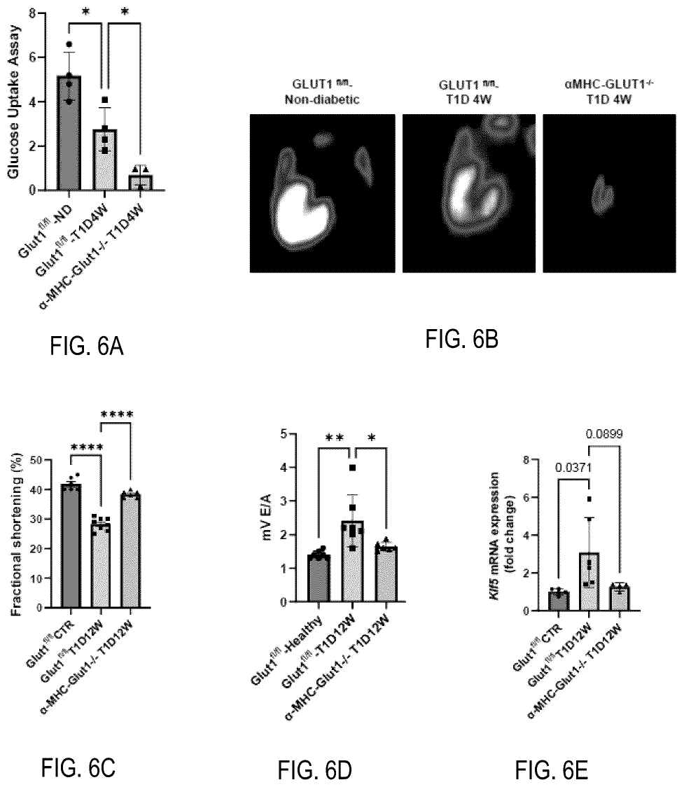

GLUT1 was inhibited genetically using cardiomyocyte-specific GLUT1 knockout mice (αMHC-GLUT1−/−). These mice confirmed the above findings regarding both type 1 and type 2 diabetes discussed in Examples 3-5. In the type 1 diabetes study, the diabetic knockout mice demonstrated a significant reduction in glucose uptake (FIGS. 6A-6B) compared with the diabetic mice with GLUT1. The diabetic knockout mice did not develop either systolic (FIG. 6C) or diastolic dysfunction (FIG. 6D). Further, KLF5 upregulation was also prevented (FIG. 6E).

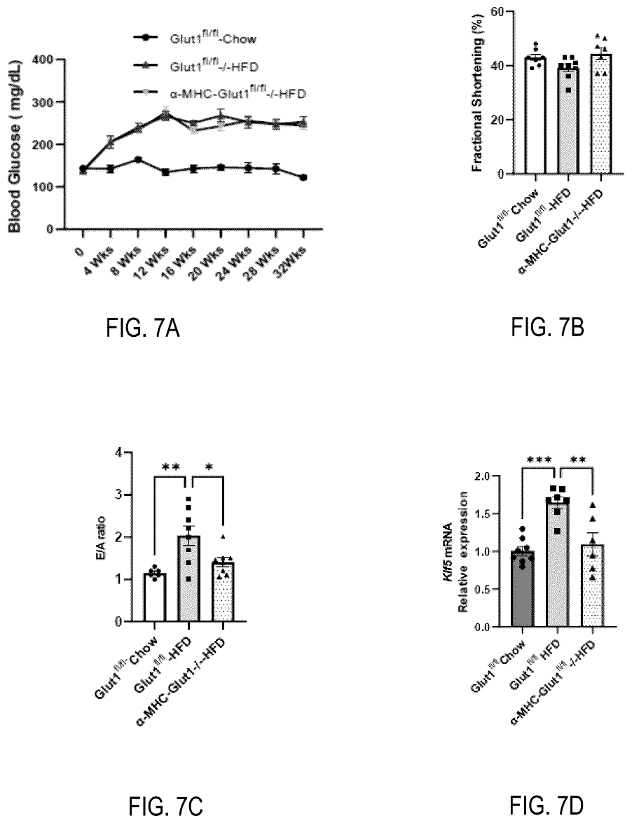

The type 2 diabetes model was repeated on the knockout mice, which similarly showed that the diabetic mice did not develop either systolic (FIG. 7B) or diastolic dysfunction (FIG. 7C) despite persistent hyperglycemia (FIG. 7A). Further, KLF5 upregulation was also prevented (FIG. 7D).

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method of treating diabetic cardiomyopathy in a patient in need thereof, the method comprising administering to the patient a therapeutically effective amount of one or more glucose transporter 1 (GLUT1) inhibitors.

2. The method according to claim 1, wherein the GLUT1 inhibitor is selected from the group consisting of STF-31, WZB117, phloretin, and BAY-876.

3. The method according to claim 1, wherein the GLUT1 inhibitor is STF-31.

4. The method according to claim 1, comprising administering the GLUT1 inhibitor systemically via an enteral or parenteral route.

5. The method according to claim 1, wherein the GLUT1 inhibitor is administered as a plurality of doses.

6. The method according to claim 1, wherein the patient is suffering from type 1 diabetes.

7. The method according to claim 1, wherein the patient is suffering from type 2 diabetes.

8. The method according to claim 1, wherein the GLUT1 inhibitor is administered after the patient is diagnosed with diabetes.

9. The method according to claim 1, wherein the GLUT1 inhibitor is administered after the patient experiences cardiac dysfunction.

10. The method according to claim 1, wherein the GLUT1 inhibitor is administered at specific time points.

11. The method according to claim 10, wherein the specific time point is selected from the group consisting of daily, weekly, or monthly.

12. The method according to claim 1, further comprising administering one or more second therapeutic agents.

13. The method according to claim 12, wherein the second therapeutic agent is selected from the group consisting of SGLT2 inhibitors, GLP1 receptor agonists, incretin mimetics, insulin, alpha-glucosidase inhibitors, biguanides, bile acid sequestrants, dopamine-2 agonists, gliptins, glinides, sulfonylurea, thiazolidinedione, and combinations thereof.

14. The method according to claim 13, wherein the second therapeutic agent is an SGLT2 inhibitor, selected from the group consisting of empagliflozin and dapagliflozin.

15. The method according to claim 13, wherein the second therapeutic agent is a GLP1 receptor agonist, selected from the group consisting of semaglutide, dulaglutide, lixisenatide, and liraglutide.

16. The method according to claim 13, wherein the second therapeutic agent is an incretin mimetics.

17. The method according to claim 13, wherein the second therapeutic agent is an insulin.

18. The method according to claim 13, wherein the second therapeutic agent is a biguanide.
